(19) United States
(12) Patent Application Publication
(10) Pub. No.: US 2023/0414224 A1
(43) Pub. Date: Dec. 28, 2023

(54) MEDICAL DEVICE RELEASE SYSTEM

(71) Applicant: Boston Scientific Scimed, Inc., Maple Grove, MN (US)

(72) Inventors: Mark Steven Smith, Coon Rapids, MN (US); Robert M. Wold, Maple Lake, MN (US); Kevin McConnell, Saint Louis Park, MN (US); Peter Benjamin Beach, Plymouth, MN (US)

(73) Assignee: BOSTON SCIENTIFIC SCIMED, INC., MAPLE GROVE, MN (US)

(21) Appl. No.: 18/214,054

(22) Filed: Jun. 26, 2023

Related U.S. Application Data
(60) Provisional application No. 63/355,857, filed on Jun. 27, 2022.

(51) Int. Cl. A61B 17/12; A61M 25/00

(52) U.S. Cl. CPC A61B 17/12145; A61M 25/0023; A61B 2017/00477

(57) ABSTRACT

A medical device system includes an elongate sheath defining a lumen extending within the elongate sheath. A coupler mechanism is slidingly disposed within the lumen, the coupler mechanism including a proximal coupler and a distal coupler. An elongate member is secured to and extends proximally from the proximal coupler. An implantable medical device (IMD) is secured to and extends distally from the distal coupler. The distal coupler remains secured to the proximal coupler while the coupler mechanism remains within the lumen, and the distal coupler releases from the proximal coupler, thereby releasing the IMD, when the coupler mechanism is exterior to the lumen.

### CROSS-REFERENCE TO RELATED APPLICATIONS

This application claims the benefit of priority of U.S. Provisional Application No. 63/355,857 filed Jun. 27, 2022, the entire disclosure of which is hereby incorporated by reference.

### TECHNICAL FIELD

The present disclosure pertains to medical devices and methods for manufacturing and/or using medical devices. More particularly, the present disclosure pertains to configurations of a system for releasing medical implants.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

The disclosure pertains to configurations of a system for releasing medical implants. An example may be found in a medical device system. The medical device system includes an elongate sheath defining a lumen extending within the elongate sheath. A coupler mechanism is slidingly disposed within the lumen, the coupler mechanism including a proximal coupler and a distal coupler. An elongate member is secured to and extends proximally from the proximal coupler. An implantable medical device (IMD) is secured to and extends distally from the distal coupler. The distal coupler remains secured to the proximal coupler while the coupler mechanism remains within the lumen, and the distal coupler releases from the proximal coupler, thereby releasing the IMD, when the coupler mechanism is exterior to the lumen.

Alternatively or additionally, the coupler mechanism may be adapted to be moved exterior to the lumen by holding the elongate member stationary while withdrawing the elongate sheath proximally.

Alternatively or additionally, the coupler mechanism may be adapted to be moved exterior to the lumen by holding the elongate sheath proximally while extending the elongate member distally.

Alternatively or additionally, the coupler mechanism may be adapted to limit relative axial movement between the distal coupler and the proximal coupler while permitting relative radial movement when not otherwise constrained by the elongate sheath.

Alternatively or additionally, the distal coupler may include a first bearing surface and the proximal coupler may include a second bearing surface, wherein the first bearing surface engages the second bearing surface to limit relative axial movement therebetween.

Alternatively or additionally, the coupler mechanism may be adapted such that distal movement of the elongate member is transmitted through the coupler mechanism to the IMD while the IMD remains secured to the distal coupler.

Alternatively or additionally, the coupler mechanism may be adapted such that proximal movement of the elongate member is transmitted through the coupler mechanism to the IMD while the IMD remains secured to the distal coupler.

Alternatively or additionally, the IMD may include an embolic coil.

Alternatively or additionally, one of the distal coupler and the proximal coupler may include a protuberance and the other of the distal coupler and the proximal coupler may include a recess complementary to the protuberance such that the protuberance fits within the recess.

Alternatively or additionally, the protuberance may be adapted to slid radially into the recess complementary to the protuberance.

Alternatively or additionally, the protuberance may include a trapezoidal protuberance.

Alternatively or additionally, the protuberance may include a rectilinear protuberance.

Alternatively or additionally, the protuberance may include a frustoconical protuberance.

Alternatively or additionally, the protuberance may include a bulbous protuberance.

Another example may be found in a system for delivering an embolic coil. The system includes an elongate sheath defining a lumen extending within the elongate sheath, an elongate member extending through the lumen, and a coupler mechanism slidingly disposed within the lumen and releasably coupling an embolic coil to the elongate member. The coupler mechanism is adapted to prevent the embolic coil from separating from the elongate member while the coupler mechanism is radially constrained by the elongate sheath, and is adapted to allow the embolic coil to separate from the elongate member when the coupler mechanism is no longer radially constrained by the elongate sheath.

Alternatively or additionally, the coupler mechanism may include a first coupler segment secured to the elongate member, and a second coupler segment secured to the embolic coil.

Alternatively or additionally, the first coupler segment may be adapted to engage the second coupler segment and limit relative axial movement therebetween when the coupler mechanism is radially constrained by the elongate sheath.

Alternatively or additionally, the first coupler segment may be adapted to disengage the second coupler segment, thereby releasing the embolic coil from the elongate member, when the coupler mechanism is not radially constrained by the elongate sheath.

Alternatively or additionally, the second coupler segment may remain secured to the embolic coil once the embolic coil has been released from the elongate member.

Another example may be found in an embolic treatment system. The embolic treatment system includes an elongate sheath defining a lumen extending within the elongate sheath, a coupler mechanism slidingly disposed within the lumen, the coupler mechanism including a proximal coupler and a distal coupler, an elongate member secured to and extending proximally from the proximal coupler, and an embolic coil secured to and extending distally from the distal coupler. The distal coupler remains secured to the proximal coupler while the coupler mechanism remains within the lumen, and releases from the proximal coupler, thereby releasing the embolic coil and the proximal coupler, when the coupler mechanism is exterior to the lumen.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a schematic side view of an illustrative delivery device for delivering an implantable medical device (IMD), showing the IMD within the delivery device;

FIG. 2 is a schematic side view of the illustrative delivery device of FIG. 1, showing the IMD expelled from the delivery device;

FIG. 3 is a schematic side view of the illustrative delivery device of FIG. 1, showing the IMD detached from the delivery device;

FIG. 4A is a perspective view of an illustrative coupler mechanism that may be used for releasably securing the IMD to the delivery device of FIG. 1;

FIG. 4B is a perspective view of the illustrative coupler mechanism of FIG. 4A, with the two coupler segments separated from each other;

FIG. 4C is a perspective view of a first coupler segment forming a part of the illustrative coupler mechanism of FIG. 4A;

FIG. 4D is a perspective view of a second coupler segment forming a part of the illustrative coupler mechanism of FIG. 4A;

FIG. 5A is a side view of an illustrative coupler mechanism that may be used for releasably securing the IMD to the delivery device of FIG. 1;

FIG. 5B is a side view of the illustrative coupler mechanism of FIG. 5A, with the two coupler segments separated from each other;

FIG. 6A is a perspective view of an illustrative coupler mechanism that may be used for releasably securing the IMD to the delivery device of FIG. 1;

FIG. 6B is a perspective view of the illustrative coupler mechanism of FIG. 6A, with the two coupler segments separated from each other;

FIG. 7A is a perspective view of an illustrative coupler mechanism that may be used for releasably securing the IMD to the delivery device of FIG. 1; and

FIG. 7B is a perspective view of the illustrative coupler mechanism of FIG. 7A, with the two coupler segments separated from each other.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

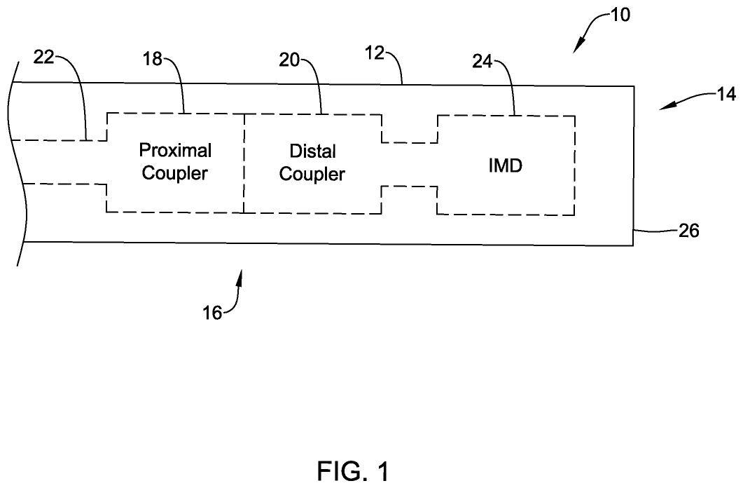

A variety of implantable medical devices (IMDs) may be delivered to a delivery site within a patient via an intravascular delivery path. FIGS. 1 through 3 provide an illustrative technique for delivering an IMD. In some cases, the IMD may be an embolic coil, but the disclosure is not limited to an embolic coil, as any of a variety of different IMDs may be delivered in the manner described herein. FIG. 1 is a schematic view of a medical device system 10 that includes an elongate sheath 12 that defines a lumen 14 extending through the elongate sheath 12. The elongate sheath 12 may be a single layer polymeric sheath, a dual layer polymeric sheath or a multiple layer polymeric sheath. The elongate sheath 12 may include reinforcing structure such as a braid or a coil, for example. In some cases, while not shown, the elongate sheath 12 may include one or more radiopaque markers that are visible under fluoroscopy, for example.

Several internal components are schematically shown in phantom. A coupler mechanism 16 is slidingly disposed within the lumen 14. In some cases, as shown, the coupler mechanism 16 includes a proximal coupler 18 and a distal coupler 20. The proximal coupler 18 may be secured to an elongate member 22 that may, for example, be manipulated at a proximal end (not shown) of the medical device system 10 in order to cause the elongate member 22, and thus the coupler mechanism 16, to move axially within the lumen 14. In some cases, the proximal coupler 18 may be secured to the elongate member 22 by laser welding, soldering, swaging, crimping or bonding a proximal end of the proximal coupler 18 to the elongate member 22. The distal coupler 20 may be secured to an IMD 24, and in some cases remains secured to the IMD 24 even after the IMD 24 has been deployed.

The proximal coupler 18 and the distal coupler 20 may be formed of any suitable material. In some cases, the proximal coupler 18 and the distal coupler 20 may be formed of platinum, iridium, stainless steel or another biocompatible material. The proximal coupler 18 and the distal coupler 20 may be manufactured using a laser ablation to ablate material from the stock being used to produce the proximal coupler 18 and the distal coupler 20. In some cases, micro machining may be used to manufacture the proximal coupler 18 and the distal coupler 20. In some cases, a combination of additive manufacturing and subtractive manufacturing may be used in creating the proximal coupler 18 and/or the distal coupler 20. Three-dimensional (3D) printing is an example of an additive manufacturing process. Ablation is an example of a subtractive manufacturing process.

In some instances, the coupler mechanism 16 may be designed such that the proximal coupler 18 and the distal coupler 20 remain secured together until such time as release of the IMD 24 is desired. For example, the proximal coupler 18 and the distal coupler 20 may be adapted to engage with each other in a manner that allows axial movement of the coupler mechanism 16 within the lumen 14 as long as the coupler mechanism 16 remains within the lumen 14. The proximal coupler 18 and the distal coupler 20 may be adapted to disengage from each other once the coupler mechanism 16, or at least a portion thereof, has moved to a position outside of the lumen 14. In some cases, the proximal coupler 18 and the distal coupler 20 are adapted to remain engaged together when the proximal coupler 18 and the distal coupler 20 are constrained from relative radial movement, including being radially constrained by the elongate sheath 12.

The coupler mechanism 16 may be adapted such that, while the coupler mechanism 16 remains within the lumen 14, axial movement of the elongate member 22 is transmitted through the coupler mechanism 16 to the IMD 24. For example, pushing the elongate member 22 in a distal direction relative to the elongate sheath 12 may cause the IMD 24 to move distally. Pulling the elongate member 22 in a proximal direction relative to the elongate sheath 12 may cause the IMD 24 to move proximally. Holding the elongate member 22 stationary while pulling the elongate sheath 12 proximally may cause a distal end 26 of the elongate sheath 12 to move closer to the IMD 24. Holding the elongate member 22 stationary while pushing the elongate sheath 12 distally may cause a distal end 26 of the elongate sheath 12 to move farther away from the IMD 24.

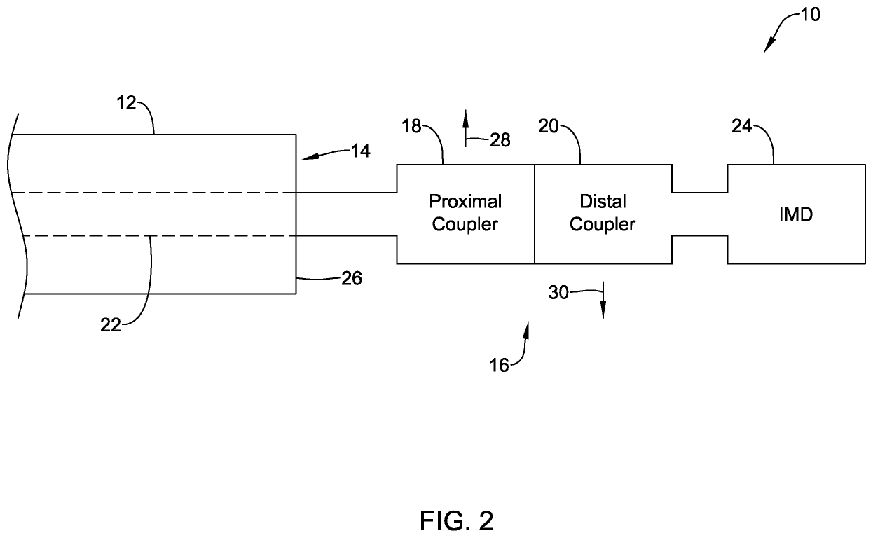

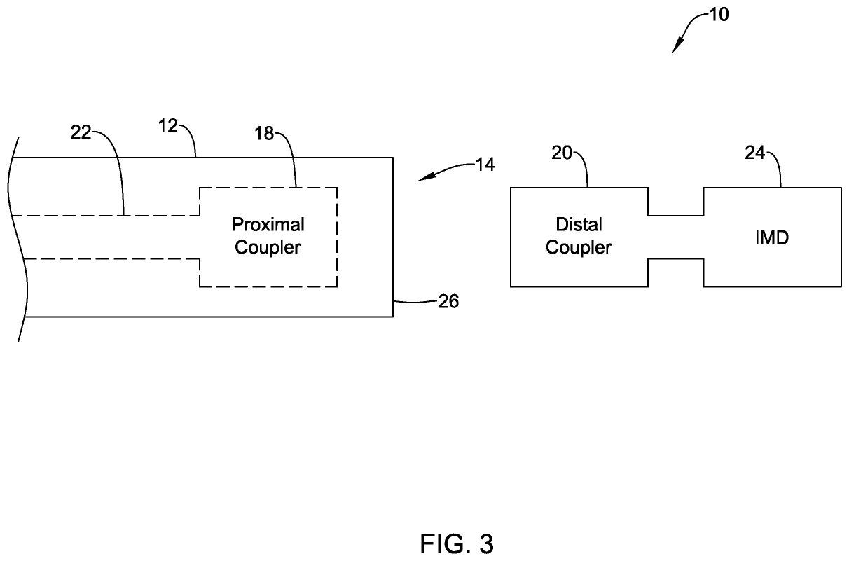

FIG. 1 shows the coupler mechanism 16 and the IMD 24 completely within the lumen 14. FIGS. 2 and 3 illustrate deployment of the IMD 24. In FIG. 2, the elongate member 22 has been advanced distally relative to the elongate sheath 12 (or the elongate sheath 12 has been withdrawn proximally relative to the elongate member 22). As a result, the coupler mechanism 16 and the IMD 24 are outside of the lumen 14, while the elongate member 22 extends proximally within the lumen 14. At this point, the coupler mechanism 16 is no longer radially constrained by the elongate sheath 12. This means that the proximal coupler 18 and the distal coupler 20 are free to move radially relative to each other. As an example, the proximal coupler 18 may be free to move in a first radial direction indicated by an arrow 28 and/or the distal coupler 20 may be free to move in a second radial direction indicated by an arrow 30, as the proximal coupler 18 may be free to move in the second radial direction indicated by the arrow 30 while the distal coupler 20 may be free to move in the first radial direction indicated by the arrow 28. In some cases, either the proximal coupler 18 and/or the distal coupler 20 may be free to move in a different radial direction not shown by the arrows 28 and 30.

Moving to FIG. 3, it can be seen that the distal coupler 20 has disengaged from the proximal coupler 18, although the distal coupler 20 remains secured to the IMD 24. In some cases, the distal coupler 20 may now be considered as being a part of the IMD 24. At this point, the elongate member 22 may be withdrawn proximally into the lumen 14 by either pulling the elongate member 22 proximally relative to the elongate sheath 12 or holding the elongate member 22 still while pushing the elongate sheath 12 distally. Next, the elongate sheath 12 (with the elongate member 22 and the proximal coupler 18) may be withdrawn from the deployment site and withdrawn from the patient. In some cases, the lumen 14 may instead be used to deliver another IMD.

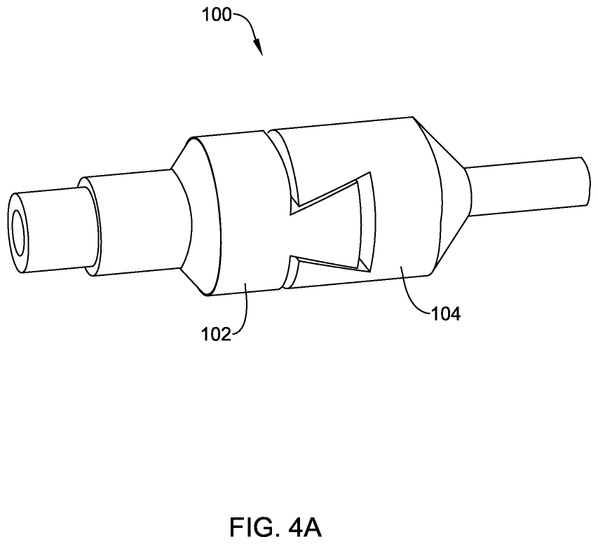

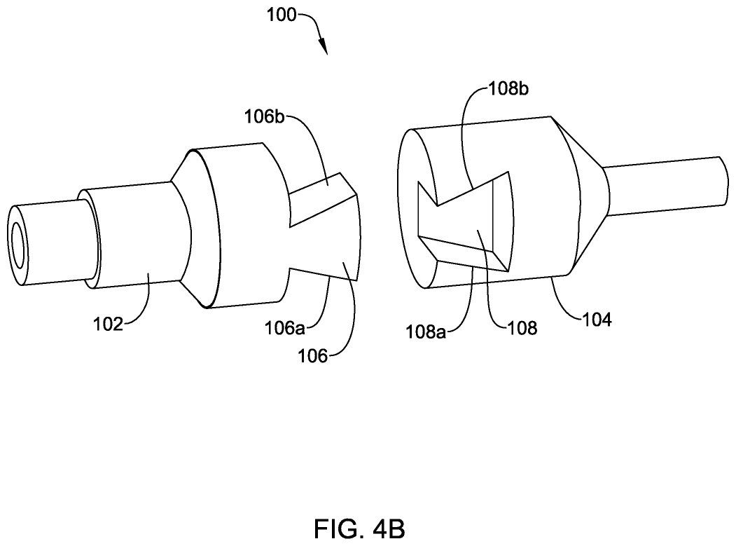

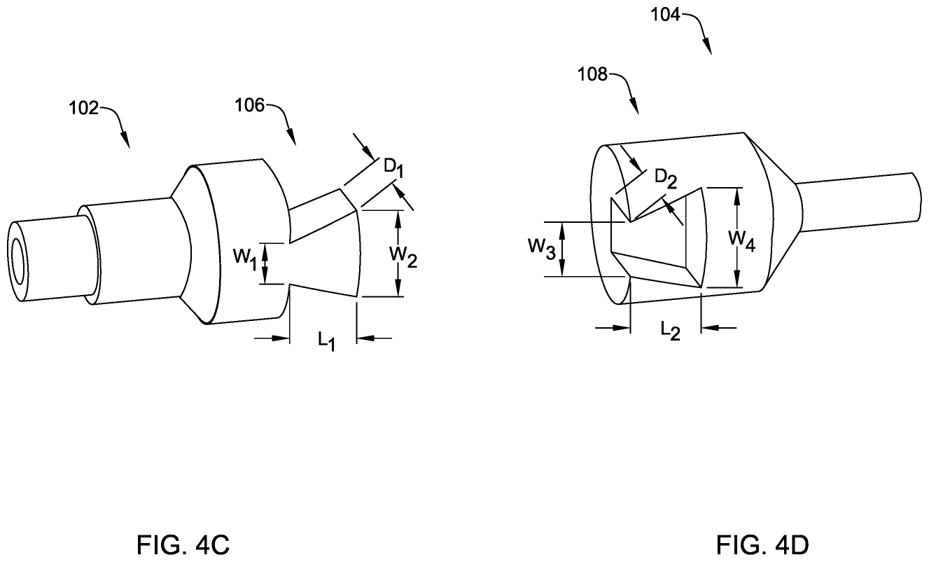

FIG. 4A is a perspective view of an illustrative coupler mechanism 100 while FIG. 4B is an exploded perspective view of the illustrative coupler mechanism 100. The illustrative coupler mechanism 100 may be considered as being an example of the coupler mechanism 16. The coupler mechanism 100 includes a first coupler segment 102 and a second coupler segment 104. In some cases, the first coupler segment 102 may be an example of the proximal coupler 18 and the second coupler segment 104 may be an example of the distal coupler 20. The IMD 24 (not shown in FIGS. 4A or 4B) may be considered as being adapted to be secured to whichever of the first coupler segment 102 and the second coupler segment 104 is functioning as the distal coupler 20. The other of the first coupler segment 102 and the second coupler segment 104 may be considered as being adapted to be secured to the elongate member 22 (not shown in FIGS. 4A or 4B).

The first coupler segment 102 includes a protuberance 106 that is adapted to fit within a corresponding recess 108 that is formed within the second coupler segment 104. In some cases, as shown, the protuberance 106 is a trapezoidal protuberance and the corresponding recess 108 is a trapezoidal recess. In some cases, the protuberance 106 includes first bearing surfaces 106a and 106b while the recess 108 includes bearing surfaces 108a and 108b. It will be appreciated that if any forces are applied to the coupler mechanism 100 that would otherwise cause the first coupler segment 102 to move axially away from the second coupler segment 104, the first bearing surfaces 106a and 106b will engage the bearing surfaces 108a and 108b in order to resist that relative axial movement.

It will be appreciated that while the drawings show crisp corners and edges, that in some cases the corners and edges may be rounded over as a result of manufacturing tolerances as well as to make it easier for components to move relative to each other. For example, to make it easier for the protuberance 106 to fit into and ultimately move radially out of the corresponding recess 108. While some features are shown as being parallel with other features, in some cases the first coupler segment 102 and the second coupler segment 104 may not be strictly formed as shown, and instead may have additional manufacturing tolerances.

FIGS. 4C and 4D are perspective views of the first coupler segment 102 and the second coupler segment 104, respectively, indicating some of the dimensions of the protuberance 106 and the recess 108. The protuberance 106, as shown in FIG. 4C, has a minimum width W₁ and a maximum width W₂. The protuberance 106 has a depth or thickness D₁ and a length Li. In some cases, W₁ may range from 0.005 inches to 0.010 inches. In some cases, W₂ may range from 0.015 inches to 0.025 inches. Di may range from 0.007 inches to 0.0175 inches. Li may range from 0.010 to 0.020 inches. In an example, Wi is 0.009 inches, W₂ is 0.019 inches, Di is inches and Li is 0.015 inches.

The recess 108, as shown in FIG. 4D, may have dimensions that are slightly larger than each of the corresponding dimensions of the protuberance 106 in order to allow the protuberance 106 to easily fit into the recess 108 in order to hold the first coupler segment 102 and the second coupler segment 104 together while allowing for easy separation when separation is desired. The recess 108 has a minimum width W₃ and a maximum width W₄. The recess 108 has a depth D₂ and a length La. In some cases, W₃ may range from 0.007 to 0.013 inches. In some cases, W₄ may range from 0.020 inches to 0.028 inches. D₂ may range from 0.007 to inches. L₂ may range from 0.012 to 0.022 inches. In an example, W₃ is 0.012 inches, W₄ is 0.022 inches, D₂ is 0.0175 inches and L₂ is 0.016 inches. These dimensions may be selected in order to ensure that there is sufficient interaction between the protuberance 106 and the recess 108 to ensure that the protuberance 106 remains within the recess 108 until such time as separation is desired.

In some cases, the first coupler segment 102 and the second coupler segment 104 may have a maximum diameter of 0.033 inches and a length of each of the first coupler segment 102 and the second coupler segment 104 is 0.030 inches. It will be appreciated that D₁ and D₂ are less than the maximum diameter. In some cases, D₁ and D₂ may be expressed as a percentage of the maximum diameter. For example, D₁ may be ten percent of the maximum diameter, or twenty percent, or thirty percent, or forty percent of the maximum diameter. D₂ may be may be ten percent of the maximum diameter, or twenty percent, or thirty percent, or forty percent of the maximum diameter, with the caveat that D₂ is at least slightly larger than D₁.

As a result, the protuberance 106 and the recess 108 are adapted such that they can only move radially apart in a specific direction. In order to separate, the first coupler segment 102 may move in a direction substantially out of the paper, relative to the second coupler segment 104, and/or the second coupler segment 104 may move in a direction substantially into the paper, relative to the first coupler segment 102, in order for the protuberance 106 to disengage from the recess 108.

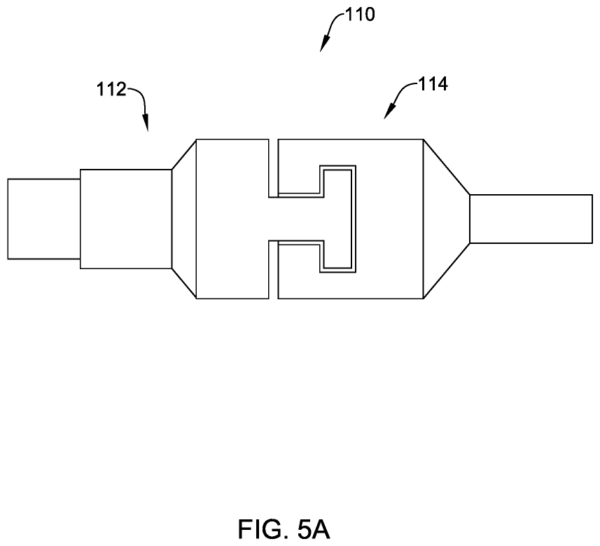

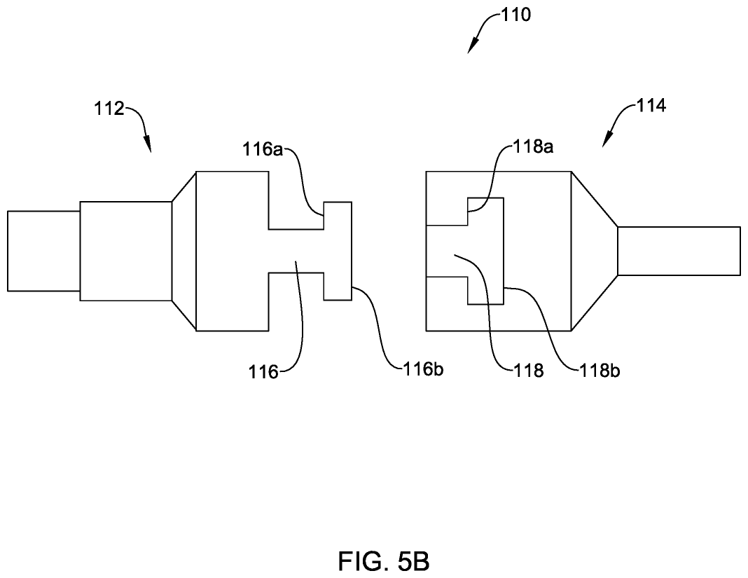

FIG. 5A is a side view of an illustrative coupler mechanism 110 while FIG. 5B is an exploded side view of the illustrative coupler mechanism 110. The illustrative coupler mechanism 110 may be considered as being an example of the coupler mechanism 16. The coupler mechanism 110 includes a first coupler segment 112 and a second coupler segment 114. In some cases, the first coupler segment 112 may be an example of the proximal coupler 18 and the second coupler segment 114 may be an example of the distal coupler 20. The IMD 24 (not shown in FIGS. 5A or 5B) may be considered as being adapted to be secured to whichever of the first coupler segment 112 and the second coupler segment 114 is functioning as the distal coupler 20. The other of the first coupler segment 112 and the second coupler segment 114 may be considered as being adapted to be secured to the elongate member 22 (not shown in FIGS. 5A or 5B).

The first coupler segment 112 includes a protuberance 116 that is adapted to fit within a corresponding recess 118 that is formed within the second coupler segment 114. In some cases, as shown, the protuberance 116 is a rectilinear protuberance and the corresponding recess 118 is a rectilinear recess. In some cases, the protuberance 116 includes an annular bearing surface 116a while the recess 118 includes an annular bearing surface 118a. It will be appreciated that if any forces are applied to the coupler mechanism 110 that would otherwise cause the first coupler segment 112 to move axially away from the second coupler segment 114, the annular bearing surface 116a will engage the annular bearing surface 118a in order to resist that relative axial movement. The protuberance 116 may be considered as including a bearing surface 116b that engages a bearing surface 118b of the recess 118 when the first coupler segment 112 is moved towards the second coupler segment 114, thereby providing pushability through the coupler mechanism 110. It will be appreciated that when not radially constrained, such as by the elongate sheath 12, the first coupler segment 112 and the second coupler segment 114 are free to move radially with respect to each other.

In some cases, and with respect to FIG. 5B, the protuberance 116 may have a diameter of 0.007 to 0.010 inches and a length of 0.007 to 0.035 inches. The bearing surface 116b may have a diameter of 0.010 to 0.018 inches and a length of 0.007 to 0.035 inches. The dimensions of the bearing surface 116 will depend upon the dimensions of the protuberance 116 and the bearing surface 116b. The dimensions of the annular bearing surface 118a will depend upon the dimensions of the recess 118 and the bearing surface 118b.

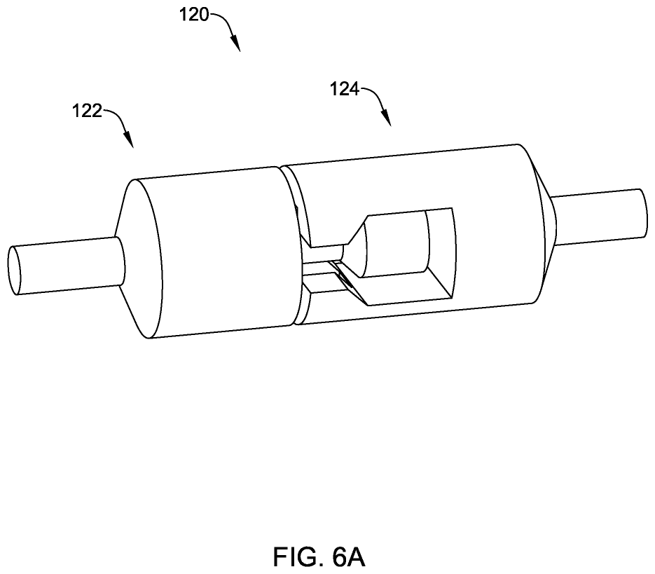

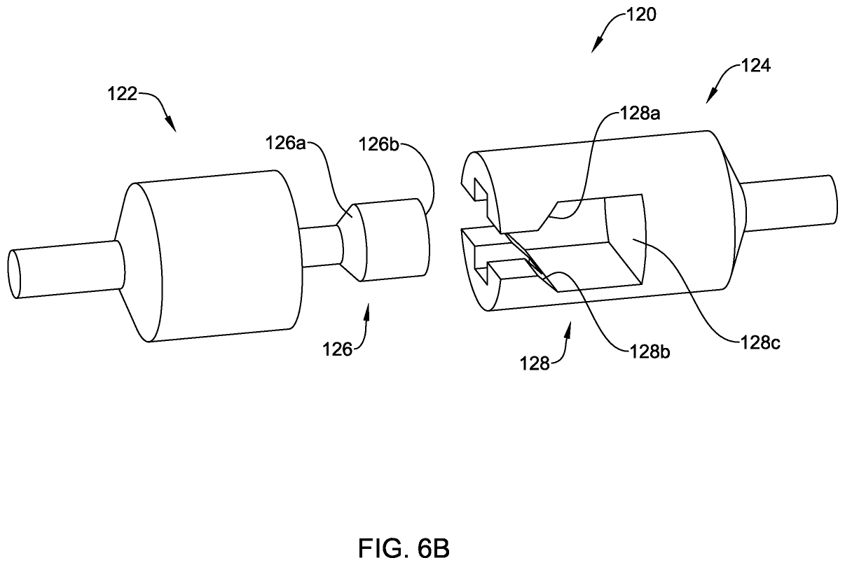

FIG. 6A is a perspective view of an illustrative coupler mechanism 120 while FIG. 6B is an exploded perspective view of the illustrative coupler mechanism 120. The illustrative coupler mechanism 120 may be considered as being an example of the coupler mechanism 16. The coupler mechanism 120 includes a first coupler segment 122 and a second coupler segment 124. In some cases, the first coupler segment 122 may be an example of the proximal coupler 18 and the second coupler segment 124 may be an example of the distal coupler 20. The IMD 24 (not shown in FIGS. 6A or 6B) may be considered as being adapted to be secured to whichever of the first coupler segment 122 and the second coupler segment 124 is functioning as the distal coupler 20. The other of the first coupler segment 122 and the second coupler segment 124 may be considered as being adapted to be secured to the elongate member 22 (not shown in FIGS. 6A or 6B).

The first coupler segment 122 includes a protuberance 126 that is adapted to fit within a corresponding recess 128 that is formed within the second coupler segment 124. In some cases, as shown, the protuberance 126 is a frustoconical protuberance and the corresponding recess 128 is adapted to accommodate the protuberance 126. In some cases, the protuberance 126 includes a conical bearing surface 126a while the recess 128 includes a first bearing surface 128a and a second bearing surface 128b. It will be appreciated that if any forces are applied to the coupler mechanism 120 that would otherwise cause the first coupler segment 122 to move axially away from the second coupler segment 124, the conical bearing surface 126a will engage the annular bearing surfaces 128a and 128b in order to resist that relative axial movement. The protuberance 126 may be considered as including a bearing surface 126b that engages a bearing surface 128c of the recess 128 when the first coupler segment 122 is moved towards the second coupler segment 124, thereby providing pushability through the coupler mechanism 120. It will be appreciated that when not radially constrained, such as by the elongate sheath 12, the first coupler segment 122 and the second coupler segment 124 are free to move radially with respect to each other.

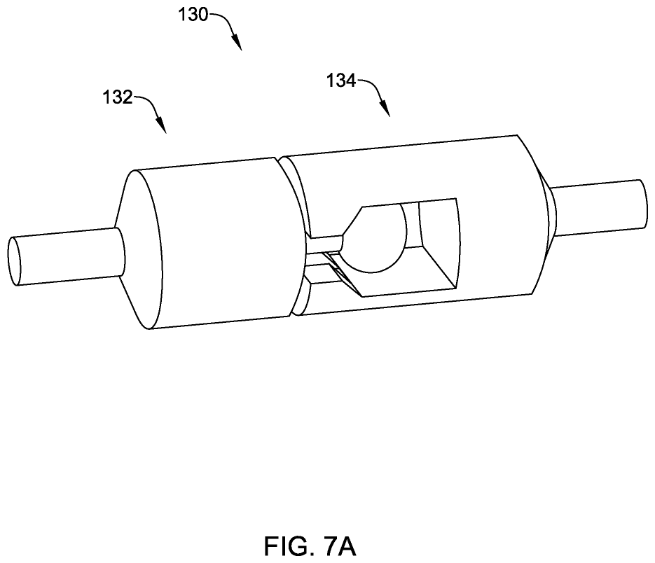

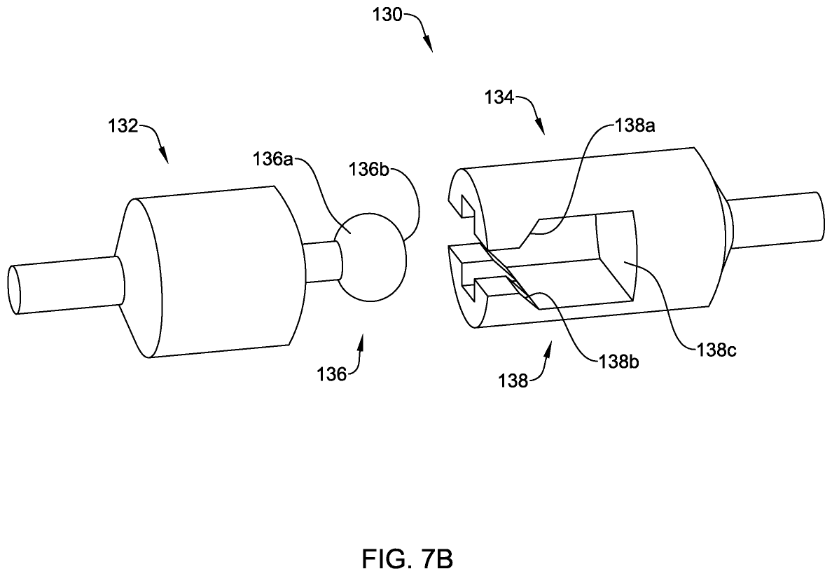

FIG. 7A is a perspective view of an illustrative coupler mechanism 130 while FIG. 7B is an exploded perspective view of the illustrative coupler mechanism 130. The illustrative coupler mechanism 130 may be considered as being an example of the coupler mechanism 16. The coupler mechanism 130 includes a first coupler segment 132 and a second coupler segment 134. In some cases, the first coupler segment 132 may be an example of the proximal coupler 18 and the second coupler segment 134 may be an example of the distal coupler 20. The IMD 24 (not shown in FIGS. 7A or 7B) may be considered as being adapted to be secured to whichever of the first coupler segment 132 and the second coupler segment 134 is functioning as the distal coupler 20. The other of the first coupler segment 132 and the second coupler segment 134 may be considered as being adapted to be secured to the elongate member 22 (not shown in FIGS. 7A or 7B).

The first coupler segment 132 includes a protuberance 136 that is adapted to fit within a corresponding recess 138 that is formed within the second coupler segment 134. In some cases, as shown, the protuberance 136 is a bulbous protuberance and the corresponding recess 138 is adapted to accommodate the protuberance 136. In some cases, the protuberance 136 includes a spherical bearing surface 136a while the recess 138 includes a first bearing surface 138a and a second bearing surface 138b. It will be appreciated that if any forces are applied to the coupler mechanism 130 that would otherwise cause the first coupler segment 132 to move axially away from the second coupler segment 134, the conical bearing surface 136a will engage the annular bearing surfaces 138a and 138b in order to resist that relative axial movement. The protuberance 136 may be considered as including a bearing surface 136b that engages a bearing surface 138c of the recess 138 when the first coupler segment 132 is moved towards the second coupler segment 134, thereby providing pushability through the coupler mechanism 130. It will be appreciated that when not radially constrained, such as by the elongate sheath 12, the first coupler segment 132 and the second coupler segment 134 are free to move radially with respect to each other.

The materials that can be used for the various components of the medical device systems described herein and the various elements thereof disclosed herein may include those commonly associated with medical devices. In some embodiments, the medical device systems described herein may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear-elastic” or “non-super-elastic” which, although may be similar in chemistry to conventional shape memory and super-elastic varieties, may exhibit distinct and useful mechanical properties. Linear-elastic and/or non-super-elastic nitinol may be distinguished from super-elastic nitinol in that the linear-elastic and/or non-super-elastic nitinol does not display a substantial “super-elastic plateau” or “flag region” in its stress/strain curve like super-elastic nitinol does. Instead, in the linear-elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super-elastic plateau and/or flag region that may be seen with super-elastic nitinol. Thus, for the purposes of this disclosure linear-elastic and/or non-super-elastic nitinol may also be termed “substantially” linear-elastic and/or non-super-elastic nitinol.

In some cases, linear-elastic and/or non-super-elastic nitinol may also be distinguishable from super-elastic nitinol in that linear-elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear-elastic materials such as stainless steel (that can also be distinguished based on its composition), which may accept only about 0.2 to 0.44 percent strain before plastically deforming.

In some embodiments, the linear-elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear-elastic and/or non-super-elastic nickel-titanium alloy. The mechanical bending properties of such material may therefore be generally inert to the effect of temperature over this very broad range of temperature. In some embodiments, the mechanical bending properties of the linear-elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear-elastic and/or non-super-elastic nickel-titanium alloy maintains its linear-elastic and/or non-super-elastic characteristics and/or properties.

In some embodiments, the linear-elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the medical device systems described herein may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the medical device systems. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device systems described herein.

In some embodiments, a degree of Magnetic Resonance Imaging (MM) compatibility is imparted into the medical device systems described herein. The medical devices described herein may be made of a material that does not substantially distort the image and create substantial artifacts (e.g., gaps in the image). Certain ferromagnetic materials, for example, may not be suitable because they may create artifacts in an MRI image. In some cases, the medical device systems, or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the medical device systems described herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the medical device systems described herein and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A medical device system, comprising:
an elongate sheath defining a lumen extending within the elongate sheath;
a coupler mechanism slidingly disposed within the lumen, the coupler mechanism including a proximal coupler and a distal coupler;
an elongate member secured to and extending proximally from the proximal coupler;
an implantable medical device (IMD) secured to and extending distally from the distal coupler;
wherein the distal coupler remains secured to the proximal coupler while the coupler mechanism remains within the lumen; and
wherein the distal coupler releases from the proximal coupler, thereby releasing the IMD, when the coupler mechanism is exterior to the lumen.

2. The medical device system of claim 1, wherein the coupler mechanism is adapted to be moved exterior to the lumen by holding the elongate member stationary while withdrawing the elongate sheath proximally.

3. The medical device system of claim 1, wherein the coupler mechanism is adapted to be moved exterior to the lumen by holding the elongate sheath proximally while extending the elongate member distally.

4. The medical device system of claim 1, wherein the coupler mechanism is adapted to limit relative axial movement between the distal coupler and the proximal coupler while permitting relative radial movement when not otherwise constrained by the elongate sheath.

5. The medical device system of claim 1, wherein the distal coupler comprises a first bearing surface and the proximal coupler comprises a second bearing surface, wherein the first bearing surface engages the second bearing surface to limit relative axial movement therebetween.

6. The medical device system of claim 1, wherein the coupler mechanism is adapted such that distal movement of the elongate member is transmitted through the coupler mechanism to the IMD while the IMD remains secured to the distal coupler.

7. The medical device system of claim 1, wherein the coupler mechanism is adapted such that proximal movement of the elongate member is transmitted through the coupler mechanism to the IMD while the IMD remains secured to the distal coupler.

8. The medical device system of claim 1, wherein the IMD comprises an embolic coil.

9. The medical device system of claim 1, wherein one of the distal coupler and the proximal coupler comprises a protuberance and the other of the distal coupler and the proximal coupler comprises a recess complementary to the protuberance such that the protuberance fits within the recess.

10. The medical device system of claim 9, wherein the protuberance is adapted to slide radially into the recess complementary to the protuberance.

11. The medical device system of claim 9, wherein the protuberance comprises a trapezoidal protuberance.

12. The medical device system of claim 9, wherein the protuberance comprises a rectilinear protuberance.

13. The medical device system of claim 9, wherein the protuberance comprises a frustoconical protuberance.

14. The medical device system of claim 9, wherein the protuberance comprises a bulbous protuberance.

15. A system for delivering an embolic coil, the system comprising:
an elongate sheath defining a lumen extending within the elongate sheath;
an elongate member extending through the lumen; and
a coupler mechanism slidingly disposed within the lumen and releasably coupling an embolic coil to the elongate member;
the coupler mechanism adapted to prevent the embolic coil from separating from the elongate member while the coupler mechanism is radially constrained by the elongate sheath;
the coupler mechanism adapted to allow the embolic coil to separate from the elongate member when the coupler mechanism is no longer radially constrained by the elongate sheath.

16. The system of claim 15, wherein the coupler mechanism comprises:
a first coupler segment secured to the elongate member; and
a second coupler segment secured to the embolic coil.

17. The system of claim 16, wherein the first coupler segment is adapted to engage the second coupler segment and limit relative axial movement therebetween when the coupler mechanism is radially constrained by the elongate sheath.

18. The system of claim 16, wherein the first coupler segment is adapted to disengage the second coupler segment, thereby releasing the embolic coil from the elongate member, when the coupler mechanism is not radially constrained by the elongate sheath.

19. The system of claim 16, wherein the second coupler segment remains secured to the embolic coil once the embolic coil has been released from the elongate member.

20. An embolic treatment system, comprising:
an elongate sheath defining a lumen extending within the elongate sheath;
a coupler mechanism slidingly disposed within the lumen, the coupler mechanism including a proximal coupler and a distal coupler;
an elongate member secured to and extending proximally from the proximal coupler;
an embolic coil secured to and extending distally from the distal coupler;
wherein the distal coupler remains secured to the proximal coupler while the coupler mechanism remains within the lumen; and
wherein the distal coupler releases from the proximal coupler, thereby releasing the embolic coil and the proximal coupler, when the coupler mechanism is exterior to the lumen.
